(19) United States
(12) Patent Application Publication
(10) Pub. No.: US 2019/0038571 A1
(43) Pub. Date: Feb. 7, 2019

(54) Orally Available Small Molecules to Inhibit the Function of the Complement System

(71) Applicants: Daniel E. Benjamin, Millstone Township, NJ (US); Frederic Houghton Brucato, Ridgefield Park, NJ (US)

(72) Inventors: Daniel E. Benjamin, Millstone Township, NJ (US); Frederic Houghton Brucato, Ridgefield Park, NJ (US)

(21) Appl. No.: 16/056,880

(22) Filed: Aug. 7, 2018

Related U.S. Application Data
(60) Provisional application No. 62/542,096, filed on Aug. 7, 2017.

(51) Int. Cl. A61K 31/09; A61P 25/18; A61K 9/00; A61K 31/724

(52) U.S. Cl. CPC A61K 31/09; A61P 25/18; A61K 31/724; A61K 9/0053; A61K 9/0014

(57) ABSTRACT

The invention relates generally to pterostilbene and a related compound thereof active to inhibit complement at concentrations lower than 1 micromole per liter in human serum. The invention also relates to methods of using these orally active, well tolerated small molecules for the treatment of Alzheimer's disease and to prevent the development of schizophrenia by inhibiting the classical complement pathway. Methods for the treatment of complement-associated inflammatory and non-inflammatory diseases, including those of the skin, are also presented.

The current application claims a priority to the U.S. Provisional Patent Application Ser. No. 62/542,096, filed Aug. 7, 2017.

### FIELD OF THE INVENTION

The invention relates generally to pterostilbene and a related compound thereof active to inhibit complement system at concentrations lower than 1 micromole per liter (1 μM) in human serum. The invention also relates to methods of using these orally active, well tolerated small molecules for the treatment of Alzheimer's disease and to prevent the development of schizophrenia by inhibiting the classical complement pathway. Methods for the treatment of complement-associated inflammatory and non-inflammatory diseases, including those of the skin, are also presented.

### BACKGROUND OF THE INVENTION

The mammalian complement system is part of the innate immune system and is involved in many functions. The complement system is composed of more than thirty different cell-surface bound or fluid phase proteins and comprises three different pathways (i.e. Classical, Lectin and Alternative pathways). The complement system helps protect against infection. Complement cascade proteins (C5, C4 C3, C2, C1) are all involved in identifying foreign antibodies and facilitate their removal (Fritzinger and Benjamin 2016).

The complement system also plays a role in developmental stages of vision, synaptic pruning, and in aging. In vision, retinal ganglion cells are subject to synaptic pruning by proteins C1q or C3 (Stevens B. et al., 2007). Without pruning of weak or defective synapses early in development, by these complement proteins, the retinogeniculate system may not develop properly. This period of active pruning coincides with the appearance of astrocytes.

Complement protein C3 appears to be involved with age-related decline in hippocampal function. Wild type mice typically show loss of neurons and synapses in hippocampal area CA3, as a function of age. However, C3 deficient mice (C3 knock-outs) show normal neuron/synaptic levels. The knockout mice also exhibit enhanced long-term potentiation (LTP) and less anxiety in a behavioral assessment of anxiety (Shi et al., 2015). Use of C3 knockout mice provides one strategy for inhibiting the complement system. In normal aging, LTP tends to fade and anxiety tends to increase. Thus, the complement system appears to play an important role in aging.

It has been shown that pharmacological inhibition of the complement system under certain conditions can prevent complement-mediated inflammation. C5aR1, the proinflammatory receptor for complement protein C5a is expressed in the CNS, on neurons, on microglia and endothelial cells. The antagonist to C5aR1, PMX205 (a cyclic hexapeptide), decreased inflammation and pathology caused by beta-amyloid in an animal model of Alzheimer's disease (Hernandez et al., 2017; Hong et al., 2016).

### Alzheimer's Disease (AD)

The etiology of Alzheimer's disease is complex and multi-factorial. Despite much effort and many clinical trials, there are no currently approved disease modifying drugs for Alzheimer's disease. However, with respect to the role of the complement system, it has been shown that the complement system initiates, in part, an immune response that removes pathogens, cellular debris [beta amyloid, ptau] and helps refine synaptic connections and neurons that may contribute to healthy aging. Specifically, complement protein C3 has been identified as an important regulator of healthy synapses as well as LTP and anxiety, an emotion affected by aging (Shi et al., 2015).

Complement C3-deficient mice fail to display age-related hippocampal decline. In two AD mouse models (Hernandez et al., 2017) blocking complement protein C5a's proinflammatory receptor, C5aR1, reduced beta amyloid pathology as well as cognitive deficits. While the antagonist PMX205 showed efficacy, an analogue PMX53, blocked beta amyloid induced loss of MAP-2. In other words, beta amyloid causes a loss of MAP2, and blocking the C5a receptor restored that loss. It is well established that accumulation of beta amyloid plaques, ptau's neurofibrillary tangles occurs in Alzheimer's disease and play a critical role in neurotoxicity. Thus, an anti-oxidant strategy was adopted early on in the fight against AD (Spagnuolo 2016). Hippocampal irradiation in young rodents produces negative effects on hippocampal development and learning. These deficits are restored in mice lacking complement protein C3 (Kalm et al., 2016). C3 deficiency ameliorates the negative effects of irradiation of the young brain on hippocampal development and learning. Irradiation of the hippocampus in young mice causes deficits in hippocampal development and learning that are ameliorated by C3 deficiency.

### Schizophrenia, Complement and Resveratrol

The psychiatric disease schizophrenia has been investigated intensively. Approximately 1.1% Americans suffer from schizophrenia. Patients followed for ten years respectively showed enduring deficits: at ten years after the first episode, 25% are recovered, and 25% are able to live independently. Another 25% need frequent care, 15% end up hospitalized, and unfortunately 10% commit suicide. Almost two million people will be diagnosed this year. The financial and emotional cost of schizophrenia is enormous, both to the families and society.

Preclinical and clinical research indicates the complement system plays an important role in development and maintenance of schizophrenia. (HAvik et al., 2011, Levite, 2014, Mayilyan et al., 2008). Inhibiting the complement system may prevent or restore aberrant neural connectivity, as well as reduce inflammation (Sekar et al., 2016). C1 receptor has been characterized in patients with schizophrenia (Arakelyan et al., 2011). Blood analysis of schizophrenic patients has also been conducted as part of the immune hypothesis of schizophrenia (Li et al., 2012; Tichaczek-Goska, 2012). In an animal model of schizophrenia, resveratrol (RSV) reduced anxiety and produced anti-psychotic effects (Magaji et al., 2017). But in at least one study, RSV did not improve cognition in schizophrenia, using measures of verbal learning, the Stroop test, the Weschler adult intelligence scale and the brief psychiatric rating scale which assessed psychopathology severity. It is known that RSV has poor bioavailability. (Zortea et al. 2016). In this study RSV failed perhaps due to its limited bioavailability (Zortea et al., 2016).

### The Prodrome Period of Schizophrenia

Schizophrenia is characterized by a pre-schizophrenia vulnerability or prodrome period, that determines, in part, the intensity and extent of the illness. The prodrome period is influenced by environmental factors, such as stress, drug use, family dynamics as well as genetic background. These factors often interact to precipitate a psychotic episode or “break” that can lead to full blown schizophrenia. The complement system may play an important role in this prodromal phase, as the complement system is affected by environment influences, as well as neurobiological substrates and genetic profiles. Complement inhibition, at the appropriate time, could modify the brain or alter neural activity such that a psychotic episode is averted in at risk individuals. Identifying genetic markers may help find individuals who are at risk for a psychotic episode and full-blown schizophrenia. Clarifying mechanisms involved with environmental factors that could influence the prodromal period is also very important. Early on-set symptoms for schizophrenia have been reported (Stentebjerg-Olesen et al., 2016).

### Identifying the Prodromal Phase

A disease modifying drug for schizophrenia would ideally be used only for at risk individuals and administered during a sensitive or vulnerable period where the potential for conversion is high. Identifying and treating an at risk individual during the prodrome period relies on a combination of approaches shown as follows:

1. Collection of behavioral data;

2. Awareness of genetic background and genetic markers, which may change during the prodrome period;

3. Advice to the individual at risk that certain behaviors and drug use may accelerate the prodrome period and increase the risk of a “break”; and

4. Pharmacological intervention.

Some of these methods are discussed in detail (Cannon, 2015), and review of behavioral changes have also been reported (Stentebjerg-Olesen et al., 2016).

Smart phone apps have been developed to help a variety of psychiatric conditions such as attention deficit hyperactivity disorder (ADHD), bipolar disorder and schizophrenia. Some of the apps are intended to help an individual monitor their behavior. In the case of schizophrenia, behavioral data collected with a smart phone app. may alert an at-risk person, such as a teen who is monitored by a physician, to activity changes consistent with a prodrome phase. The physician may wish to watch more closely and/or formulate a treatment plan. The smart phone approach may also be valuable to patients with well established schizophrenia. For a schizophrenic patient, a relapse is both damaging and demoralizing, says Dr. Ben-Zeey, an assistant professor of psychiatry at Dartmouth College and Smart phone study's principal investigator. An unmonitered, at risk for schizophrenia patient, or schizophrenic patient, could face jail, hospitalization, or possibly suicide. The Cross Check study proposed by Dr. Ben-Zeey will include 150 schizophrenic patients who have been discharged from Zucker Hillside Hospital, in Glen Oaks, within the past year. Half of those patients will receive smartphones with the app, while the other half, the control group, will receive the standard clinical services that the hospital provides after a patient is discharged. The study will look at the time lag between when they're recruited and their first relapse, and also at the number of relapses they have over a year.”

Priori is a smartphone app created by researchers at the University of Michigan, that will attempt to spot the early signs of mood swings in people with bipolar disorder. So far researchers have trained the app to recognize manic and depressive moods by analyzing the user's voice, tracking pitch as well as patterns of speech and silence. The next iteration of Priori will use these subtle signals to prompt an intervention, such as a call from a therapist.

### Pharmacological Intervention

There is some interest in antidepressant use for prodrome schizophrenia. While results of this study were mixed, clearly this approach represents a potentially valuable approach towards treating at risk kids and averting full-blown schizophrenia (Cornblatt et al., 2007) as well as approaches that include drug treatment (Millan et al., 2016).

Environmental Factors that May Increase Risk During the Prodrome Period

One of environment factors that may increase risk during the prodrome period is drug use. There is a substantial amount of data that indicates teenagers at high risk for conversion are at even greater risk from the effects of marijuana (cannabis). Marijuana consumed chronically acts as an agonist at the CB 1 receptor, creating a constellation of effects, possibly including altered dopamine and glutamate activity, increased cortisol and interaction with genetic markers such as DISC1 (Brucato 2017a; Brucato 2017b; Carol et al., 2017; Dragt et al., 2012; Ballinger et al., 2015).

Some investigators have speculated that the subjective “marijuana high” experienced by teens, may shift in at risk teens, leading to anxious periods, strong dysphoria and potential derealization moments. These affective changes could signal an at risk teen is entering a shifting prodromal period. Understanding this dynamic interaction between marijuana, genetic profile, and prodromal shift could lead to better timing and treatment during prodrome.

Previous studies have shown that resveratrol (RSV) protects CA1 neurons from ischemia reperfusion damage (Li et al., 2016). It has also been demonstrated that resveratrol (RSV) and blueberry extract improve spatial memory and cognition in a rat model of AD (Wang et al., 2017; Dal-Pan et al., 2017; Chen et al., 2017). Resveratrol has also been shown to ameliorate renal damage and to have anti-complement, anti-oxidative and anti-apoptotic effects in a murine model of membranous nephropathy (WU et al., 2015). While there is a dearth of information supporting RSV's positive health effects, RSV's utility is limited by its poor bioavailability for therapeutic applications. Thus, there is a need to develop some other stilbene-based compounds such as pterostilbene and its related compounds for therapeutic application.

### SUMMARY OF THE INVENTION

The following listing of embodiments is a non-limiting statement of various aspects of the invention. Other aspects and variations will be evident in light of the entire disclosure.

Some embodiments include a method of inhibiting the complement system in blood, brain, or peripheral organs comprising administering an effective amount of pterostilbene or a related compound thereof to a patient, wherein the patient is a human or an animal. In one embodiment, the method is used to treat a variety of diseases such as Alzheimer's or any other brain and nerve disease at any stage in the patient. In another embodiment, the method is used to treat prodromal schizophrenia that prevents the development of schizophrenia in a at risk patient, or, the method is used to prevent relapse in successfully treated schizophrenia. These diseases can be ameliorated by inhibiting complement activity. In some embodiments, the pterostilbene or a related compound thereof is taken orally, inhaled, topically applied, and parenterally administered to the patient to reach an end organ concentration between 0.2 to 30 μM to inhibit complement in the brain and periphery. In one embodiment, the pterostilbene or a related compound thereof inhibits the classical complement cascade. In another embodiment, the pterostilbene or a related compound thereof inhibits C4 and C2 (C1q, C1r, C1 s) convertase activity, or C2a convertase activity in the method. In other embodiments, the pterostilbene or a related compound thereof inhibits C3 (C4b2a) convertase activity, C5 (C3b₂Bb) convertase activity, complement Factor B activity, and/or complement Factor D activity. In some embodiments, the pterostilbene or a related compound thereof inhibits the complement-mediated synaptic pruning.

Some embodiments include a method of inhibiting the classical complement cascade in blood, brain, or peripheral organs comprising orally administering an effective amount of pterostilbene or a related compound thereof to a patient, wherein the patient is a human or an animal. In one embodiment, the method is used to ameliorate neuroinflammatory diseases selected from the group consisting of neuromyelitis optica, multiple sclerosis, and amyotrophic lateral sclerosis (ALS) in the patient. In another embodiment, the method is used to ameliorate an inflammatory disease consisting of rheumatoid arthritis in the patient.

Some embodiments include a method of inhibiting the classical complement cascade in blood, brain, or peripheral organs comprising topically administering an effective amount of pterostilbene or a related compound thereof alone or in combination with other useful compounds to a human or an animal that has disorders of the skin. In one embodiment, the method is used to treat cannabis or tetrahydrocannabinol overdose or intoxication in the patient.

Some embodiments include a method of inhibiting the classical complement cascade in blood, brain, or peripheral organs comprising topically or orally administering an effective amount of pterostilbene or a related compound thereof alone or in combination with beta-cyclodextrin to a human or an animal that has internal or brain disorders. In one embodiment, the method is used to treat cannabis or tetrahydrocannabinol overdose or intoxication in the patient.

### BRIEF DESCRIPTION OF THE DRAWINGS

FIG. 1. Inhibition of complement-mediated sheep erythrocyte lysis by CCB-695 (Pterostibene). Various concentrations of pterostilbene, ranging from 240 nM to 50 uM were added to normal human serum, which was then incubated at 37° C. with sensitized sheep erythrocytes.

### DETAIL DESCRIPTION OF THE INVENTION

While some information supports RSV's positive health effects, RSV's utility is limited by its poor bioavailability for therapeutic applications. However, RSV's analogue pterostilbene (Ptero, structure shown below) is very promising due to its superior pharmacokinetics and bioavailability (Kapetanovic et al., 2011). Indeed, much of the work with RSV is now being repeated with Ptero. Ptero's superior pharmacokinetics, oral bioavailability, and metabolic profile along with increased potency raises the possibility that it may act as a disease modifying drug for diseases such as Alzheimer's disease, schizophrenia, amyotrophic lateral sclerosis (ALS), Parkinson's Disease and others. The invention disclosed here involves the use of pterostilbene (a natural product) or a related compound thereof (stilbene-based compound with different substituents on the benzene ring) to inhibit the complement system. The inhibition is at the level of C1q association with C1r and C1s to create C2 and C4 convertase activity, and inhibition of the convertase. This invention also relates to inhibiting the C3 convertase or C5 convertase with stilbene-based drugs and natural products.

### Chemicals Claimed as Stilbene-Based Complement Inhibitors

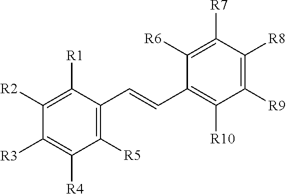

| R1 | R2 | R3 | R4 | R5 | R6 | R7 | R8 | R9 | R10 |
|---|---|---|---|---|---|---|---|---|---|
| H | H | H | H | H | H | H | H | H | H |
| OH | OH | OH | OH | OH | OH | OH | OH | OH | OH |
| OCH₃ | OCH₃ | OCH₃ | OCH₃ | OCH₃ | OCH₃ | OCH₃ | OCH₃ | OCH₃ | OCH₃ |
| CF₃ | CF₃ | CF₃ | CF₃ | CF₃ | CF₃ | CF₃ | CF₃ | CF₃ | CF₃ |

Molecules with the general stilbene structure, where the 10 substituents can have either H, OCH₃, CF₃, or OH. For example, pterostilbene (Ptero) has a stilbene structure with R2 and R4 being OCH₃, R8 being OH, and the rest of substituents being H. Resveratrol (RSV) has a stilbene structure with R2, R4 and R8 being OH and the rest of substituents being H. Both pterostilbene and resveratrol are commercially available and can be found in natural sources such as blueberries. Some of stilbene-based compounds with OH, OCH₃ or CF₃ substituent can be prepared via organic synthesis (Savouret et al., 2004). Each position could also be substituted with a S or a glucuronide group that suits the needs for activity or physicochemical properties for a given application.

The term “a” or “an” means more than one. For example, pterostilbene or “a” related compound thereof, the term “a” means more than one related compound of pterostilbene. The term “a related compound thereof” means a compound like pterostilbene has a stilbene-based structure with a substituent of H, OCH₃, CF₃, or OH, or, a S or a glucuronide group, and the related compound also has similar activity to pterostilbene such as inhibitory activity against the complement system.

### Example 1: Inhibition of Complement-Mediated Sheep Erythrocyte Lysis by Pterostibene (CCB-695)

The sheep erythrocyte lysis assay is a commonly used assay to demonstrate complement activity (Ahmed et al., 2015, White et al., 2010). To demonstrate the activity of the stilbene molecules, we added them to normal human serum before incubating the serum with activated sheep erythrocytes. The lysis of the sheep erythrocytes is mediated by the classical complement pathway.

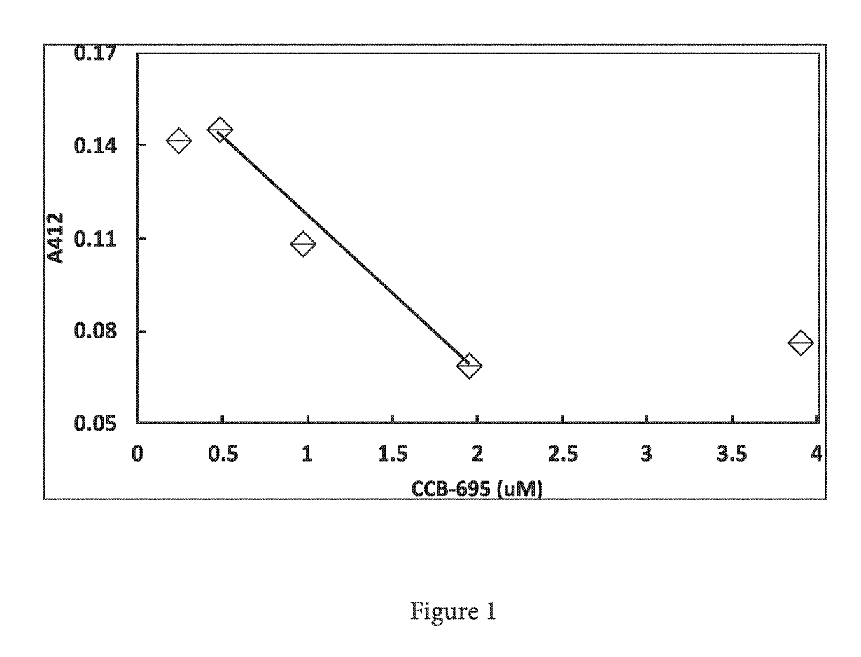

FIG. 1 shows inhibition of complement-mediated sheep erythrocyte lysis by CCB-695 (Pterostilbene). Various concentrations of pterostilbene, ranging from 240 nM to 50 uM were added to normal human serum, which was then incubated at 37° C. with sensitized sheep erythrocytes. After a 30 min incubation, the remaining unlysed erythrocytes were pelleted by centrifugation and the amount of hemoglobin released by cell lysis was quantified by absorption spectroscopy at wavelength of 412 nm. Inhibition of lysis indicates reduced complement activity. This is representative data from 3 separate experiments. Resveratrol was also active in this assay but exhibited only one third the potency.

The natural product pterostilbene, a putative complement inhibitor, shows greater potency than resveratrol (RSV) in a sheep erythrocyte lysis assay [Benjamin, unpublished 2017]. This fact that pterostilbene inhibits complement, yet most likely preserves RSV's anti-oxidant, anti-inflammatory and neuroprotective characteristics and shows greater bioavailability and potency as a complement inhibitor, increases the therapeutic value of pterostilbene. This information also positions pterostilbene as a disease modifying drug for schizophrenia. If at risk individuals are treated with pterostilbene during a schizophrenia prodrome period, pterostilbene may prevent schizophrenia from ever occurring. It would do this by acting on the complement system and/or many of the mechanisms listed above.

All illustrations of the drawings are for the purpose of describing selected versions of the present invention and are not intended to limit the scope of the present invention.

Although the invention has been explained in relation to its preferred embodiment, it is to be understood that many other possible modifications and variations can be made without departing from the spirit and scope of the invention as hereinafter.

What is claimed is:

1. A method of inhibiting the complement system in blood, brain, or peripheral organs comprising administering an effective amount of pterostilbene or a related compound thereof to a patient, wherein the patient is a human or an animal.

2. The method according to claim 1, wherein the method is used to treat a variety of diseases that occur in the patient and the diseases can be ameliorated by inhibiting complement activity.

3. The method according to claim 1, wherein pterostilbene or a related compound thereof is taken orally, inhaled, topically applied, and parenterally administered to reach an end organ concentration between 0.2 to 30 μM to inhibit the complement system in the brain and periphery.

4. The method according to claim 1, wherein pterostilbene or a related compound thereof inhibits the classical complement cascade.

5. The method according to claim 1, wherein pterostilbene or a related compound thereof inhibits C4 and C2 (C1q, C1r, Cis) convertase activity.

6. The method according to claim 1, wherein pterostilbene or a related compound thereof inhibits C2a convertase activity.

7. The method according to claim 1, wherein pterostilbene or a related compound thereof inhibits C3 convertase (C4b2a) activity.

8. The method according to claim 1, wherein pterostilbene or a related compound thereof inhibits C5 convertase (C3b₂Bb) activity.

9. The method according to claim 1, wherein pterostilbene or a related compound thereof inhibits the complement Factor B activity.

10. The method according to claim 1, wherein pterostilbene or a related compound thereof inhibits the complement Factor D activity.

11. The method according to claim 1, wherein pterostilbene or a related compound thereof inhibits the complement-mediated synaptic pruning.

12. The method according to claim 11, wherein the method is used to treat Alzheimer's or any other brain and nerve disease at any stage in the patient.

13. The method according to claim 11, wherein the method is used to treat prodromal schizophrenia that prevents the development of schizophrenia.

14. The method according to claim 11, wherein the method is used to prevent relapse in successfully treated schizophrenia.

15. A method of inhibiting the classical complement cascade in blood, brain, or peripheral organs comprising orally administering an effective amount of pterostilbene or a related compound thereof to a patient, wherein the patient is a human or an animal.

16. The method according to claim 15, wherein the method is used to ameliorate neuroinflammatory diseases selected from the group consisting of neuromyelitis optica, multiple sclerosis, and amyotrophic lateral sclerosis in the patient.

17. The method according to claim 15, wherein the method is used to ameliorate an inflammatory disease consisting of rheumatoid arthritis in the patient.

18. A method of inhibiting the classical complement cascade in blood, brain, or peripheral organs comprising topically administering an effective amount of pterostilbene or a related compound thereof alone or in combination with other useful compounds to a human or an animal that has disorders of the skin.

19. A method of inhibiting the classical complement cascade in blood, brain, or peripheral organs comprising topically or orally administering an effective amount of pterostilbene or a related compound thereof alone or in combination with beta-cyclodextrin to a human or an animal that has internal or brain disorders.

20. The method according to claim 19, wherein the method is used to treat cannabis or tetrahydrocannabinol overdose or intoxication in the patient.
